(19) United States
(12) Patent Application Publication
(10) Pub. No.: US 2008/0254535 A1
(43) Pub. Date: Oct. 16, 2008

(54) Cell Separation Apparatus

(76) Inventors: Ryosuke Takahashi, Kawagoe (JP); Kazutoshi Kan, Kawagoe (JP); Toyoshige Kobayashi, Hatoyama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 12/027,323

(22) Filed: Feb. 7, 2008

(30) Foreign Application Priority Data
Apr. 12, 2007 (JP) 2007-104386

(51) Int. Cl. C12M 1/00

(52) U.S. Cl. 435/308.1

(57) ABSTRACT

A conventional method of retrieving spheroids formed needs to chemically label the spheroids and may physically damage the spheroids. An apparatus according to the present invention has a nanopillar cell culture sheet, a flow speed generating section, and a spheroid retrieval section. The present invention uses the nanopillar cell culture sheet to provide a mechanism that first forms spheroids, a mechanism that provides a flow speed to the spheroids formed, and a mechanism that retrieves the spheroids released owing to the provided flow speed, without the need for labeling.

### CLAIM OF PRIORITY

The present application claims priority from Japanese application JP 2007-104386 filed on Apr. 12, 2007, the content of which is hereby incorporated by reference into this application.

### BACKGROUND OF THE INVENTION

1. Field of the Invention

The present invention relates to a method of forming a tissue or a tissue-like structure containing cells or cell masses (spheroids) and a method of retrieving the tissue or the tissue-like structure, and to a method for forming a spheroid, a mechanism that provides the formed spheroids with a flow speed, and a container in which spheroids released owing to the provided flow speed are retrieved; the method, the mechanism, and the container are useful for, for example, regenerative medicine.

2. Background Art

Cell culture experiments have generally been conducted in two-dimensional culture dishes. On the basis of knowledge obtained through the experiments, cell culture techniques have been significantly improved to date. In contrast, cells and tissues in a multicellular organism contact not only one another but also a complicated assembly of biopolymers (mesh structure) such as a basement membrane or an extracellular matrix (ECM) that support the cells and tissues. That is, in the organism, the cells and tissues exist in the three-dimensional structure. Therefore, a Matrigel which is a cell culture system mimicking such an in vivo environment as described above, a method for culturing cells by implanting cells in a collagen gel, and so on have been developed. Among them, the Matrigel, which is a structure obtained by solubilizing a basement membrane matrix containing extracted materials from an ECM such as collagen, has been used for a cancer cell invasion assay modeling, an angiogenesis analysis modeling using vascular endothelial cells, and the like, which has provided much knowledge that has not been acquired through culture systems with simple two-dimensional planes such as culture dishes, significantly contributed to development in this field. It is also known that, culture in a three-dimensional environment produces more similar results for gene expression to those of in vivo gene expressions compared to a two-dimensional culture.

In recent years, attempts have been made to apply a nanoimprint microfabrication technique to scaffold materials for cell culture or tissue culture. A “nanopillar sheet” has been developed which has a plurality of projections each having a nanoscale diameter and a height that is dozens of times the diameter (for example, JP Patent Publication (Kokai) No. 2004-170935 A). The nanopillar sheet is a functional substrate comprising a group of pillar-like microprojections which are made of an organic polymer and the position, bottom area, and height of which can be controlled. Efforts have been made to apply the nanopillar sheet to the fields of semiconductor devices, optical components, storage devices, and the like. Moreover, the use of the nanopillar sheet as a cell culture container has been reported (for example, JP Patent Publication (Kokai) No. 2005-312343 A). The nanopillar cell culture sheet has an advantage in that the artificially designed micro three-dimensional structure can be used as a scaffold material to solve the above-described problems with the Matrigel and it is expected to be effectively used as a three-dimensional culture device. It has actually been found that the nanopillar has a specific effect on cells (for example, The official journal of The Japanese Society for Regenerative Medicine, 5: 91-95 (2006)).

On the other hand, various important reports have been made, indicating the excellence of the spheroid culture system as an in vitro cell culture system. In several examples, the spheroid culture system has been applied to cell types such as liver cells (for example, Biochem. Byophys. Res. Comm. 322: 684-692 (2004)). Although the spheroid culture system is an excellent culture system as described above, disadvantageously, neither a method for simply forming spheroids nor a method for simply retrieving the spheroids formed has been established. A method has been proposed which forms spheroids on a cell non-adhesive base material surface using a cell culture base material having cell adhesive domains of the order of micrometers and retrieves the spheroids in a noninvasive manner (for example, JP Patent Publication (Kokai) No. 2006-67987 A). However, this method requires complicated operations of applying the cell adhesive domains during formation and culturing the cells using a PBS containing no divalent metal ions during retrieval.

### SUMMARY OF THE INVENTION

The clinical application of cultured cells and tissues has a very high utility value provided that a simple method is available for forming spheroids. The utility value is further increased if any technique is available for retrieving the spheroids formed without the need for chemical labeling and without physically damaging the spheroids.

The Matrigel is a very effective experimental system. However, the Matrigel product varies with the batch and cannot be customized for a target experiment.

The method described in Biochem. Byophys. Res. Comm. 322: 684-692 (2004) is an example in which the spheroid culture system is applied to liver cells. In the example, with plane culture, the amount of albumin produced started to decrease four days after the beginning of the experiment. In contrast, with spheroid culture, the amount of albumin produced increased continuously for the first six days and the production subsequently continued. However, no method has been developed for retrieving the spheroids thus obtained, which have albumin production activity.

The method described in JP Patent Publication (Kokai) No. 2006-67987 A describes the method of producing spheroids and the method of retrieving the spheroids produced. However, the methods require the complicated operations of producing spheroids utilizing the substrate with cell adhesive domains applied thereto and culturing cells using the PBS not containing divalent metal ions during retrieval.

On the other hand, the present inventors have found that spheroids are likely to be formed depending on the low adhesiveness of the nanopillar sheet.

Thus, an object of the present invention is to produce and retrieve spheroids only through physical effects using a nanopillar cell culture sheet. A further object of the present invention is to provide a mechanism that forms spheroids, a mechanism that provides the formed spheroids with a flow speed, and a mechanism that retrieves spheroids released owing to the provided flow speed without using labeling.

The present inventors have found that cells are likely to form spheroids on a nanopillar sheet. The present invention provides the following configuration. Cells or spheroids are produced on a nanopillar sheet having a plurality of pillar-like microprojections. A flow speed generating section is coupled to a container containing the nanopillar sheet therein; the flow speed generating section is made up of a driving section, a control section, a syringe pump, and a discharge opening which cooperatively generate a liquid flow. The container is provided with the flow speed through the discharge opening to release the cells or spheroids from the nanopillar sheet. The spheroids are retrieved in a spheroid retrieval section.

One example of apparatus according to the present invention has a projecting member including a plurality of pillar-like microprojections, a container in which the projecting member and a liquid are housed, and a control section allowing a liquid flow of the liquid to be generated inside the container.

The spheroids can be easily formed on the nanopillar sheet, and the spheroids produced can be retrieved by the very simple method, that is, only through the physical effects. The present invention does not require any reagent such as a spheroid formation promoting agent, the treatment of the bottom surface of a cell culture dish, or the like. This reduces damage to the spheroids. Moreover, the spheroids can be separated into groups according to the size of each spheroid. That is, spheroids of a greater or smaller diameter can be selectively retrieved and applied to the field of regenerative medicine or the like.

### BRIEF DESCRIPTION OF THE DRAWINGS

FIG. 1 shows the configuration of an apparatus described in Example 1;

FIG. 2 shows the average values of the diameters of spheroids measured after a separation operation;

FIG. 3 is an image drawing showing conditions generated before and after the separation operation;

FIG. 4 shows the number of cells observed after 4-day culture of spheroids remaining on a nanopillar after the separation operation and released and retrieved spheroids, all the spheroids being subjected to a trypsin treatment and then seeded before the culture;

FIG. 5 shows the configuration of an apparatus described in Example 2;

FIG. 6 shows the configuration of an apparatus described in Example 3; and

FIG. 7 shows an example of a nanopillar sheet.

The present invention will be further illustrated in the following examples. In the examples, embodiments of the present invention used for the culture of spheroids will be described as an example. However, the present invention is also applicable to cells that do not form any spheroids or tissues or tissue-like structures containing spheroids.

### EXAMPLE 1

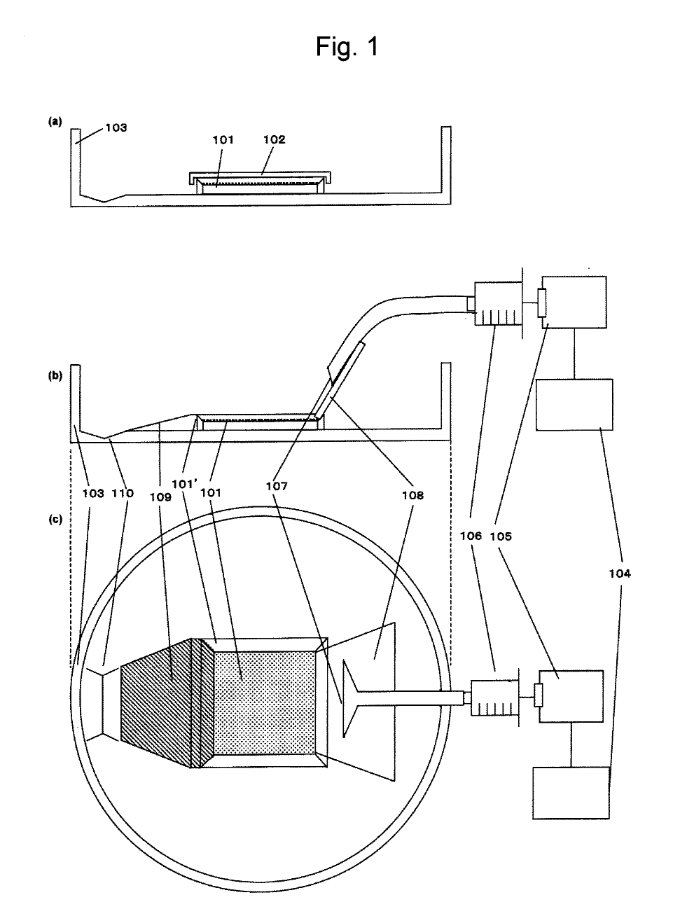

An example of the present invention will be described with reference to FIG. 1. FIGS. 1(a) and (b) show an apparatus as viewed from the side thereof. FIG. 1(c) shows the apparatus as viewed from above. FIG. 1(a) shows how the apparatus is configurated during culture of spheroids, and FIGS. 1(b) and (c) show how the apparatus is configurated during retrieval of spheroids. The present apparatus has a nanopillar sheet section (101) serving as a substrate on which spheroids are formed.

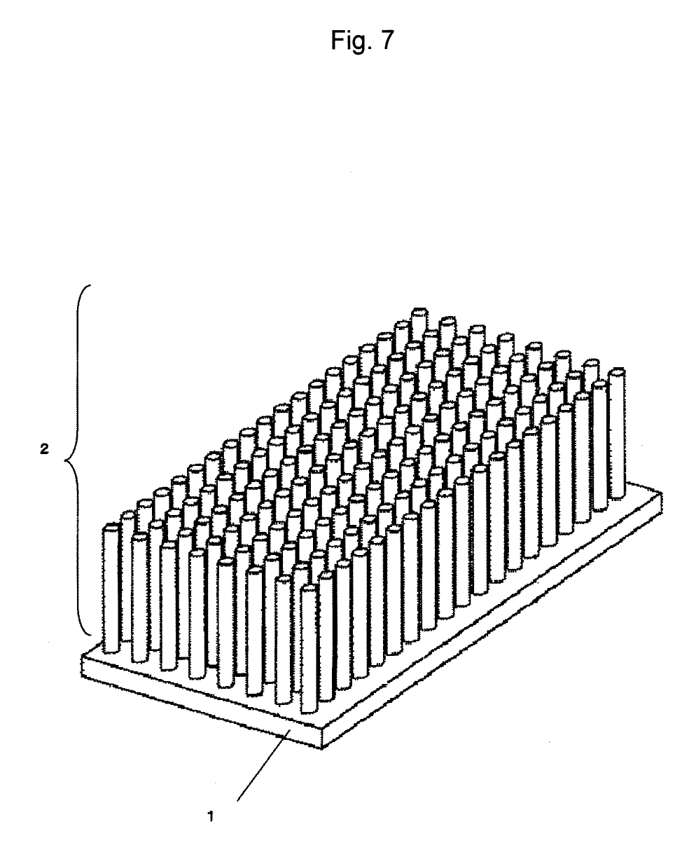

The nanopillar sheet refers to a projecting member having a base material 1 composed of a thermoplastic organic polymer and a group of pillar-like microprojections 2 extending from a base material as shown in FIG. 7 by way of example. In particular, the projections constituting the group of pillar-like microprojections may have an equivalent diameter of 10 nm to 500 μm and a height of 50 nm to 5,000 μm. The nanopillar sheet section (101) may be square or rectangular. The nanopillar sheet section (101) is shaped like a wall that is higher than the group of pillar-like microprojections at an end (101′) of the base material. The group of pillar-like microprojections is surrounded by the wall in four directions to enable a liquid culture medium to be held. Cells are cultured here and a cover (102) is set over the nanopillar sheet section during the culture. The nanopillar sheet section is located inside a container (103).

To retrieve spheroids formed, the cover (102) is removed and a control section (104), a driving section (105), and a syringe pump (106) which cooperatively generate a liquid flow are arranged outside the container holding the nanopillar sheet section (101). A liquid flow from a syringe pump (106) is provided to the nanopillar sheet section (101) through a tube via a discharge opening (107) and thus to the spheroids formed on the nanopillar sheet section (101) for retrieval. The control section (104) and the driving section (105) can set the flow speed of the liquid. A first plate-like member (diffusion plate) (108) is provided below the discharge opening (107) to substantially uniformly provide the liquid flow to the nanopillar sheet section. The first plate-like member is located at an angle of greater than 0 degrees to the nanopillar sheet section. Some of the spheroids provided with the liquid flow overflow from the nanopillar sheet section and are collected in a spheroid retrieval section (110) through a second plate-like member (inclined plate) (109) located at an angle of greater than 0 degrees to the nanopillar sheet section. The spheroid retrieval section (110) is located adjacent to the nanopillar sheet section and downstream of the nanopillar sheet section in the direction of the liquid flow.

On the other hand, spheroids not overflowing in spite of the liquid flow remain on the nanopillar sheet section (101). The spheroid retrieval section is recessed in the center thereof. The overflowing spheroids are thus collected in this part and can be easily retrieved.

An example of an experiment using the apparatus shown in FIG. 1 will be described below. A V79, a Chinese hamster lung fibroblast-like cell line, was used as a material. The cell line was seeded on the nanopillar sheet sections (101) each of a pillar diameter of 0.5 μm, 1.0 μm, or 2.0 μm at 1.0×10⁵ cells/ml and cultured for 72 hours. A DMEM (SIGMA) with an FBS (ICN) added so as to set a final concentration at 10% was used for culture. After the formation of spheroids was confirmed, liquid flows of 173 μl/s, 246 μl/s, and 492 μl/s through the syringe pump (106) were provided via the discharge opening (107) on the diffusion plate (108) to retrieve the spheroids.

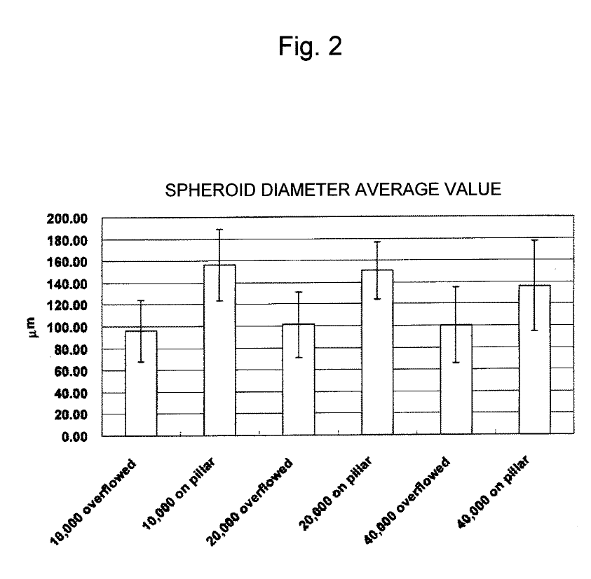

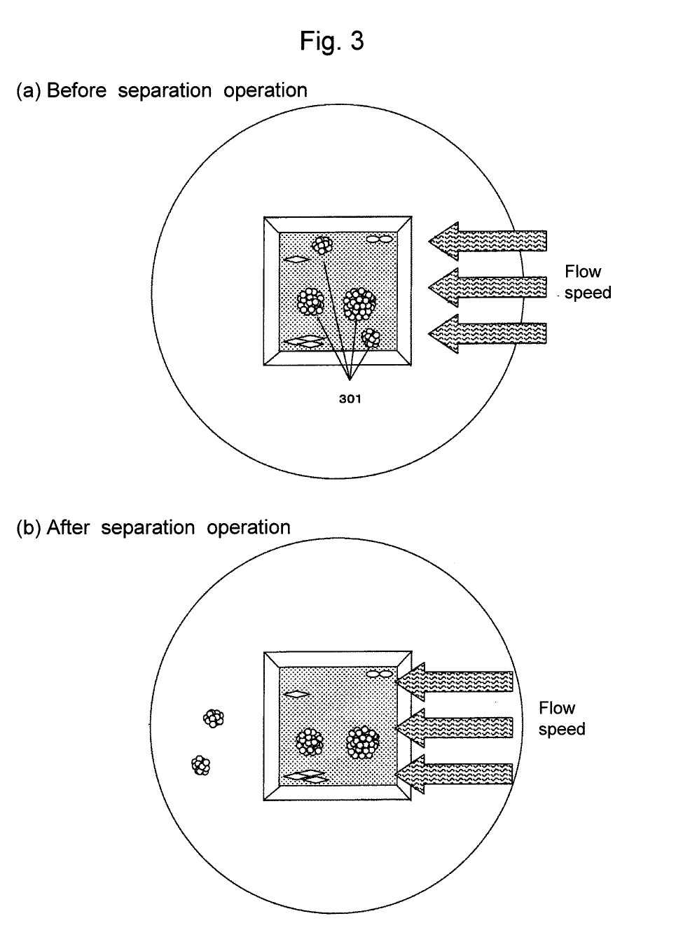

As a result, a portion of the spheroids were retrieved in the spheroid retrieval section (110), with the others remaining on the nanopillar sheet section. After the retrieval operation, the diameter of the spheroids retrieved in the spheroid retrieval section was compared with that of the spheroids remaining on the nanopillar sheet section. The results are shown in FIG. 2. The spheroids retrieved in the spheroid retrieval section (110) had a diameter of 80 μm regardless of the flow speed. In contrast, the spheroids remaining on the nanopillar sheet section had a diameter of greater than 100 μm on average. A schematic diagram of the results is shown in FIG. 3. FIG. 3(a) shows a situatuion before the separation operation is performed. FIG. 3(b) shows a situation after the separation operation. The figures show that among the the spheroids formed (301), larger ones remain on the nanopillar sheet, whereas smaller ones are separately retrieved in the spheroid retrieval section. This indicates that the spheroids can be separated into groups depending on a difference in diameter in this method.

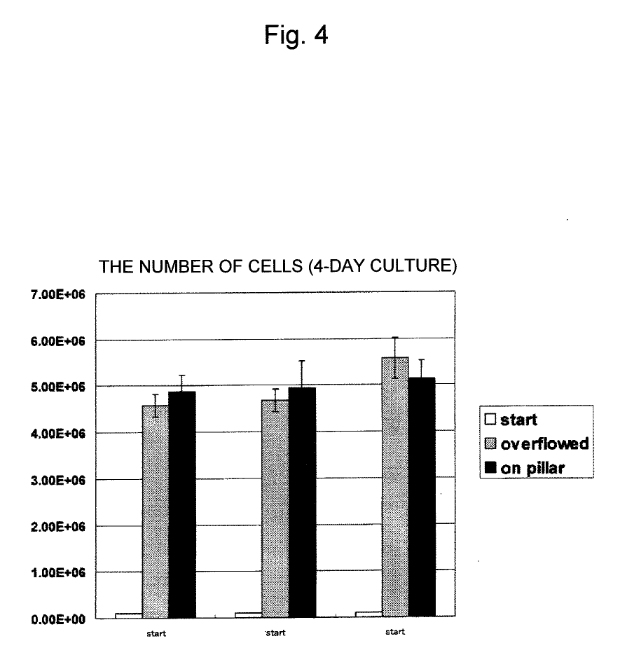

Furthermore, a trypsin solution was used to treat the spheroids remaining on the nanopillar sheet after the retrieval operation and the spheroids released from the nanopillar sheet and successfully retrieved. The cells were separated from one another, re-seeded in a culture dish, and cultured for four days to check the cells for proliferative ability. The results are shown in FIG. 4. The number of resultant cells was compared with that of cells present at the beginning of the culture (see, “start” in FIG. 4). No difference in proliferative ability per unit time was detected between the cells constituting the spheroids remaining on the nanopillar sheet (see, “on pillar” in FIG. 4) and the cells constituting the spheroids released from the nanopillar sheet (see, “overflowed” in FIG. 4) regardless of the flow speed (FIG. 4). This indicates that the separation operation according to the present invention does not damage the cells.

### EXAMPLE 2

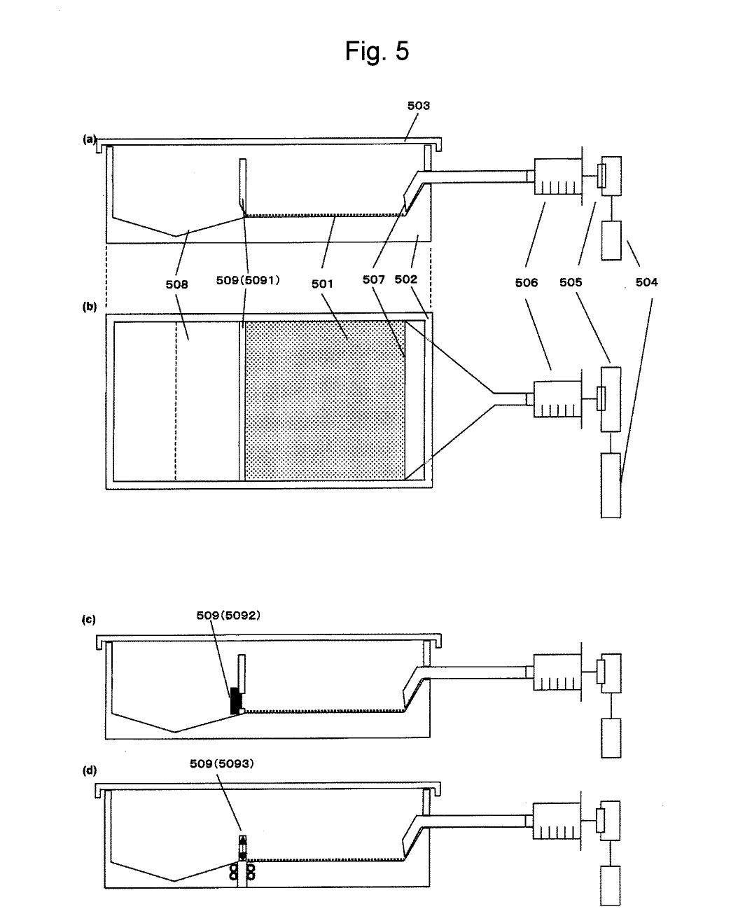

Another example will be illustrated with reference to FIG. 5. FIGS. 5(a), (c), and (d) are diagrams showing examples of apparatuses as viewed from the side thereof. FIG. 5(b) is a diagram of one of the apparatuses as viewed from above. Each of the apparatuses has a nanopillar sheet section (501) serving as a substrate on which spheroids are formed. The nanopillar sheet section (501) may be square or rectangular. The nanopillar sheet section (501) is located in a container (502) and can thus hold a liquid culture medium. Cells are cultured on the nanopillar sheet section (501). A cover is denoted by 503 (FIGS. 5(a), (c), and (d) show an apparatus of which the cover is set on the container, and FIG. 5(b) shows an apparratus of which the cover has been removed). A control section (504), a driving section (505), and a syringe pump (506) which cooperatively generate a liquid flow are arranged outside the container (502) holding the nanopillar sheet section. A liquid flow from the syringe pump (506) is provided to the nanopillar sheet section (501) through a tube via a discharge opening (507) and thus to the spheroids formed on the nanopillar sheet section (501) for retrieval. The control section (504) and the driving section (505) can define the flow speed of the liquid.

The liquid flow allows the spheroids to be retrieved in the spheroid retrieval section (508). At this time, a partition (509) located opposite the discharge opening (507) must be removed. This mechanism may be such that a retrieval path is established by cutting a plate-like member (5091) located so as not to contact the nanopillar sheet section and serving as a partition, releasing a joint member (in this case, rubber packing) fixing the bottom of the partition during spheroid formation (5092 in FIG. 5(c)), or moving the partition upward and downward (5093 in FIG. 5(c)). A portion of the spheroids subjected to a liquid flow overflow from the nanopillar sheet section and are collected in the spheroid retrieval section (508). On the other hand, spheroids not overflowing in spite of the liquid flow remain on the nanopillar sheet section (501). The spheroid retrieval section (508) is recessed in the center thereof. The overflowing spheroids are thus collected in this part and can be easily retrieved.

By thus providing the partition allowing the liquid flow path to be installed, between the nanopillar sheet section and the spheroid retrieval section, the location of the liquid can be easily controlled both during the spheroid growth stage and during the spheroid retrieval stage even when large nanopillar sheet area is set up in the container. In the present example, the spheroids need not climb over a wall as used with conventional nanopillar sheets before the spheroids can reach the spheroid retrieval section. This allows the spheroids to be more efficiently retrieved.

### EXAMPLE 3

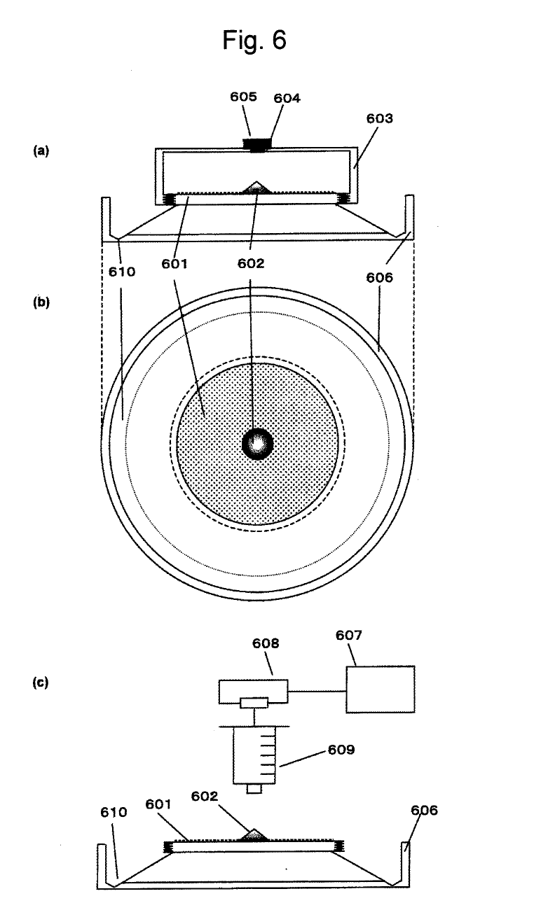

An example of the present invention will be illustrated with reference to FIG. 6. FIGS. 6(a) and (c) are diagrams showing an apparatus as viewed from the side thereof. FIG. 6(b) is a diagram of the apparatus as viewed from above. The apparatus has a substantially circular nanopillar sheet section (601) on which spheroids are formed and which holds a substantially conical structure (conical portion 602) in a substantial center thereof. During cell culture (a), a threaded cover (603) can be set over the nanopillar sheet section (601) to hold a liquid culture medium on the nanopillar sheet section (601) for cell culture. A hole (604) is formed in a central part of the cover so as to allow a cell suspension to be added to the culture medium through the hole. During culture, the hole can be blocked with rubber packing (605), this substrate is located in a container (606) that houses the substrate. To retrieve spheroids formed (as shown in FIG. 6(b)), the cover is removed. A control section (607), a driving section (608), and a syringe pump (609) which cooperatively generate a liquid flow are arranged outside the container (606), holding the nanopillar sheet section. A liquid from a liquid flow from the syringe pump (609) located above the nanopillar sheet section is dropped onto a vertex of a conical part (602) or the vicinity of the vertex to generate a liquid flow. The liquid flow is concentrically provided to the nanopillar sheet section (601). The provided liquid flow allows the spheroids to be retrieved in the spheroid retrieval section (610). An inclined area is provided between the nanopillar sheet section and the spheroid retrieval section (610). This inclination allows the released spheroids to be collected in the spheroid retrieval section (610) for easy retrieval.

### INDUSTRIAL APPLICABILITY

The main cell culture has hitherto been single-layer culture on a two-dimensional plane. However, since the in vivo environment is three-dimensional, it is indispensable to produce a cell culture system mimicking the environment and a cell group having a three-dimensional structure. The present invention not only enables spheroids, cell masses having such a three-dimensional structure, to be easily formed but also allows the spheroids to be easily retrieved. The present invention thus has an industrial applicability particularly in the field of regenerative medicine.

What is claimed is:

1. An apparatus comprising:
a projecting member comprising a plurality of pillar-like microprojections;
a container in which the projecting member and a liquid are housed; and
a control section allowing a liquid flow of the liquid to be generated inside the container.

2. The apparatus according to claim 1, further comprising a retrieval section located adjacent to the projecting member.

3. The apparatus according to claim 1, further comprising a retrieval section located downstream of the projecting member in a direction of the liquid flow.

4. The apparatus according to claim 2, wherein the projecting member holds cells contacting the pillar-like microprojections, and the retrieval section retrieves the cells.

5. The apparatus according to claim 1, wherein the projecting member comprises a thermoplastic organic polymer.

6. The apparatus according to claim 1, wherein the projecting member is higher than each of the pillar-like microprojections at an end of the projecting member.

7. The apparatus according to claim 1, further comprising a first plate-like member located between the control section and the projecting member at an angle of greater than 0 degree to the projecting member.

8. The apparatus according to claim 2, further comprising a second plate-like member located between the projecting member and the retrieval section at an angle of greater than 0 degree to the projecting member.

9. The apparatus according to claim 1, wherein the control section controls the liquid flow such that the liquid flow is substantially uniformly provided to the projecting member.

10. The apparatus according to claim 2, further comprising a partition which is located between the projecting member and the retrieval section and which allows a liquid flow path to be installed.

11. The apparatus according to claim 1, wherein a substantially conical structure is provided in a substantial center of the projecting member.

12. The apparatus according to claim 11, wherein the control section controls the liquid flow such that the liquid is dropped onto a vertex of the structure or a vicinity of the vertex.
